(19) United States
(12) Patent Application Publication
(10) Pub. No.: US 2004/0175700 A1
(43) Pub. Date: Sep. 9, 2004

(54) Method for cohort selection

(75) Inventor: Bard J. Geesaman, Cambridge, MA (US)

Correspondence Address: FISH & RICHARDSON PC, 225 FRANKLIN ST, BOSTON, MA, 02110, US

(73) Assignee: ELIXIR PHARMACEUTICALS, INC.

(21) Appl. No.: 10/378,397

(22) Filed: Mar. 3, 2003

(51) Int. Cl. C12Q 1/68; G01N 33/53; G06F 19/00; G01N 33/48; G01N 33/50

(52) U.S. Cl. 435/6; 435/7.1; 702/19; 702/20

(57) ABSTRACT

Information for each individual of a first group of individuals and each individual of a second group of individuals is used to select a subset of individuals from the second group. The information can be about a plurality of different biological features. The selection can use a comparison between information for members of the first group and information for members of the subset. It is also possible to compare members of the first group to members of the selected subset with respect to at least one factor. The method can be used to reduce stratification, for example, in the analysis of genetic associations.

### BACKGROUND

Genetic association studies are used to identify genetic markers and genes associated with a particular trait. Typically, genetic information is obtained from individuals that have the particular trait (the “cases”) and is compared with genetic information from control individuals that do not have the particular trait (the “controls”) or have the trait to a different degree. Multiple hypotheses are generated that test whether genetic markers are over or under represented in the case individuals compared to the control individuals.

However, some genetic association studies have been plagued by both false positive and false negative results (type I and type II error, respectively). One recognized problem is a failure to adequately match the genetic background of the cases and controls. This phenomenon is referred to as stratification. Stratification can arise, for example, in a study of a genetic trait where individuals in the class being studied are non-randomly distributed with respect to a particular genetic background. Alleles that are associated with that genetic background, but that are not causative for the trait can be erroneously associated with the trait.

### SUMMARY

In one aspect, the invention features a method that analyzes information that uses information for each individual of a first group of individuals and each individual of a second group of individuals. The information for each individual comprises indications about a plurality of different biological features. The method includes selecting a subset of individuals from the second group using a comparison between information for members of the first group (or a subset of thereof) and information for members of the subset; and evaluating the relationship of at least one factor to members of the first group relative to members of the selected subset. In a machine based implementation, at least part of the information can be received, e.g., from a user, or from instrumentation that analyzes a biologic. The method can also include outputting (e.g., displaying, sending, storing, or transmitting) a result of the comparing, e.g., to a user, a computer, a memory, and so forth.

In one embodiment, the different biological features can include at least one property of a biomolecule, e.g., a protein, nucleic acid, lipid, or carbohydrate. For example, the property of the biomolecule relates to one or more of: nucleic acid sequence, DNA methylation state, DNA accessibility, transcription factor binding, protein sequence, protein structure, protein conformation, protein aggregation state, protein localization, post-translational modification, mRNA sequence, mRNA structure, mRNA localization, mRNA chemical modification, carbohydrate structure, carbohydrate sequence, membrane composition, membrane fluidity, and so forth.

In one embodiment, the different biological features include a property of a cell, e.g., cell differentiation state, cell size, cell number or abundance, mitotic index, divisional state, gene expression state, metabolic state, extracellular-associated molecules, tissue localization, and so forth. In one embodiment, the different biological features include a property of an organism, e.g., anatomical features, blood pressure, pigmentation (hair, eye, skin). In some embodiments, the different biological features include various combinations of properties about biomolecules, cells, and organisms. The plurality of different biological features can also be restricted to features of exclusively one category, e.g., features only about post-translational modifications, or only about nucleic acid sequence.

For example, the different biological features can include information about a plurality of genetic polymorphisms, e.g., an indication of presence or absence of at least one polymorphism at a genetic locus, e.g., an indication about the presence or absence of a minor or major allele. In one embodiment, the features include an indication of presence or absence of a minor allele and a corresponding indication for the major allele. This allelic information can be phased or unphased. In one embodiment, the polymorphisms include one or more of: a SNP, RFLP, a repeat sequence, a transposon, a retroviral sequence (e.g., LTR), a microsatellite marker (e.g., LINE or SINE), insertion, deletion, substitution, or inversion. In one embodiment, the polymorphism is a biallelic polymorphism. In another embodiment, the polymorphism is a multiallelic polymorphism.

The plurality of different biological features can include at least some quantitative features. The plurality of different biological features can include at least some qualitative features. The plurality of different biological features can include at least some features that are represented by a binary variable.

In one embodiment, the plurality of different biological features includes at least five or ten features, e.g., between 10-500, 20-200, or 50-100 features.

The comparison can include use of a model (e.g., a Bayesian network or information theory model) or a comparative function. In one embodiment, the comparison includes representing the information for each member as a multi-dimensional vector or matrix.

In one embodiment, the comparison is weighted by covariance of at least two different features, e.g., by a covariance matrix for at least some or all features of the plurality of different features.

In one embodiment, the selecting includes selecting a subset that compares to the first group more favorably than at least another subset, e.g., more favorably than average or median or more favorably than at least 70, 80, 90, 95% of other possible subsets, e.g., most favorably.

In one embodiment, the selecting includes incrementally adding members of the second group to the subset. For example, the incremental adding is repeated until the subset contains the same number of members as the first group.

In one embodiment, the incremental adding includes selecting a single member of the second group based on how a group that includes the single member (e.g., the single member plus the previous selected subgroup) compares to the first group. For example, the incremental adding includes selecting a single member of the second group that minimizes a comparative function for a comparison between a group that includes the single member (e.g., the single member plus the previous selected subgroup) and the first group.

In another embodiment, the incremental adding includes selecting a cluster of members of the second group based on how a group that includes the cluster (e.g., the cluster plus the previous selected subgroup) compares to the first group. For example, the incremental adding includes selecting a cluster of members of the second group that minimizes a comparative function for a comparison between a group that includes the cluster (e.g., the cluster plus the previous selected subgroup) and the first group.

In another embodiment, the selecting includes pairing each member of the first group to a unique member of the second group. The pairing can include evaluating a comparative function. The pairing can include identifying a member of the second group that compares most favorably to the respective member of the first group.

In one embodiment, the comparison includes a comparative function that returns a value, e.g., a scalar or multivariate value. The selecting can include minimizing the comparative function.

For example, the comparative function can be a function of distance. The distance can be weighted, e.g., for genetic (e.g., allelic) variability, variance, and co-variance. The distance can be a function of a Euclidean distance, z-score distance, Bhattacharya distance, Mahalanobis distance, Matusita distance, divergence metric, Chernoff distance, angular metric, Earth Mover's distance, Hausdorff distance, City Block (Manhattan) distance, Chebychev distance, Minkowski distance, or Canberra distance. In another example the comparative function is a function of a statistical test, e.g., the mean chi-square of the G-test or a one minus Pearson correlation.

In another embodiment, the comparison includes assessing similarity using neural networks, Bayesian networks, support vector machines, or information theory.

In one embodiment, multiple subsets are selected.

In one embodiment, the individuals are animals. In another embodiment, the individuals are plants. In still another embodiment, the individuals are protists. Typically, the individuals are all from the same species.

The evaluating of the relationship of at least one factor to members of the first group relative to members of the selected subset can include determining a statistical association of the factor among members of the first group relative to members of the selected subset. For example, the factor can be a feature common to at least 30, 50, 70, 80, 90, or 95% of members of the first group. For example, the factor can be a genetic polymorphism or other biological feature.

In another aspect, the invention features a method that includes: obtaining nucleic acid samples from each individual of a plurality of individuals, wherein a first group of the individuals are associated with a trait and a second group of individuals are not associated with the trait; analyzing the nucleic acid samples to determine genetic information about a plurality of genetic loci for each individual of the plurality; selecting a subset of individuals from the second group based on a comparison between the genetic information for members of the first group and the genetic information for members of the subset; and evaluating association of a genetic locus of interest and individuals of the first group relative to association of the genetic locus of interest and individuals of the selected subset. The method can be used, for example, to evaluate the relationship between a genetic polymorphism and a trait.

For example, the genetic information includes an indication of presence or absence of at least one polymorphism at a genetic locus, e.g., an indication about the presence or absence of a minor or major allele. In one embodiment, the genetic information includes an indication of presence or absence of a minor allele and a corresponding indication for the major allele. The genetic information can be phased or unphased.

In one embodiment, the polymorphism is a SNP, RFLP, a repeat sequence, a transposon, a retroviral sequence (e.g., LTR), a microsatellite marker (e.g., LINE or SINE), insertion, deletion, substitution, or inversion. In one embodiment, the polymorphism is a biallelic polymorphism. In another embodiment, the polymorphism is a multiallelic polymorphism.

In one embodiment, the selecting includes selecting a subset that compares to the first group more favorably than at least another subset, e.g., more favorably than average or median or more favorably than at least 70, 80, 90, 95% of other possible subsets, e.g., most favorably.

In one embodiment, the selecting includes incrementally adding members of the second group to the subset. For example, the incremental adding is repeated until the subset contains a particular number of members relative to the size of the first group, e.g., the same number of members as the first group. In another example, the incremental adding is repeated until no additional members of the second group can be identified which can be added to the selected subset without exceeding a threshold value.

In one embodiment, the incremental adding includes selecting a single member of the second group based on how a group that includes the single member (e.g., the single member plus the previous selected subgroup) compares to the first group. For example, the incremental adding includes selecting a single member of the second group that minimizes a comparative function for a comparison between a group that includes the single member (e.g., the single member plus the previous selected subgroup) and the first group.

In another embodiment, the incremental adding includes selecting a cluster of members of the second group based on how a group that includes the cluster (e.g., the cluster plus the previous selected subgroup) compares to the first group. For example, the incremental adding includes selecting a cluster of members of the second group that minimizes a comparative function for a comparison between a group that includes the cluster (e.g., the cluster plus the previous selected subgroup) and the first group.

In another embodiment, the selecting includes pairing each member of the first group to a unique member of the second group. The pairing can include evaluating a comparative function. The pairing can include identifying a member of the second group that compares most favorably to the respective member of the first group.

In one embodiment, the comparison includes a comparative function that returns a value, e.g., a scalar or multivariate value. The selecting can include minimizing the comparative function.

For example, the comparative function can be a function of distance. The distance can be weighted, e.g., for genetic (e.g., allelic) variability, variance, and co-variance. The distance can be a function of a Euclidean distance, z-score distance, Bhattacharya distance, Mahalanobis distance, Matusita distance, divergence metric, Chernoff distance, angular metric, Earth Mover's distance, Hausdorff distance, City Block (Manhattan) distance, Chebychev distance, Minkowski distance, or Canberra distance. In another example the comparative function is a function of a statistical test, e.g., the mean chi-square of the G-test or a one minus Pearson correlation.

In another embodiment, the comparison includes assessing similarity using neural networks, Bayesian networks, support vector machines, or information theory.

In one embodiment, multiple subsets are selected.

In one embodiment, the evaluating of the association includes evaluating a LOD score for one or more genetic loci (e.g., polymorphic markers) of interest. The plurality of genetic markers can exclude the marker of interest. In one embodiment, the plurality of genetic markers contains between 5-500, 5-200, 10-100, or 10-80 different markers or at least 5, 10, 20, 30 or 50 markers, or less than 500, 200, 100, 80, or 50 markers. The plurality of genetic markers can be preselected, e.g., randomly selected or selected, e.g., to distribute over two or more chromosomes (e.g., at least 5, 10, 12, or 18 chromosomes), to distribute between various distances from a centromere or telomere, to include various degrees of heterozygosity, or to exclude one or more regions of interest (e.g., suspect regions).

The method can further include obtaining information about each individual of the plurality, e.g., medical information about each individual. The method can include examining each individual, e.g., for a trait, symptom, disease, or other discernable phenotype. Examining can include invasive and non-invasive (e.g., imaging techniques). For example, the individuals are humans. The method can include interviewing the individual (e.g., about medical history, family history, environmental exposure, behavior, social, or societal perceptions, etc.)

The information can include information about one or more symptoms for a disease of interest.

The second group is typically larger than the first group. For example, the second group includes at least 0.2, 0.5, 1.0, 1.5, 2, 2.5, 5, or 10 times more members than the first group. The selected subset can be any size relative to the first group, e.g., the same size, or within 10, 20, or 30% of the size of the first group, e.g., larger or smaller than the first group.

The selecting can include using more than one comparison, e.g., in addition to a first comparison, filtering a result using a second comparison. For example, the selecting can include filtering the results using a statistical test, e.g., the mean chi-square of the G-test. In one embodiment, the selecting includes a filter that requires that the mean chi-square of the G-test is less than 1.5.

The method can include other features described herein.

In another aspect, the invention features a method that includes: obtaining DNA samples from each individual of a first group of individuals and each individual of a second group of individuals; analyzing the DNA samples to determine information about a plurality of genetic markers for each individual of the first and second groups; selecting a subset of individuals from the second group using a comparison between the information for members of the first group and the information for members of the subset; and comparing members of the first group to members of the selected subset with respect to at least one factor.

In one embodiment, the comparing can include subjecting members of the first group, but not the second group to a condition and evaluating members of the first group and members the second group. For example, the condition is a medical procedure (e.g., a therapeutic or diagnostic procedure) (e.g., a drug regimen, a diet, a physical therapy plan, a psychological treatment and so forth). In another example, the condition is a behavior or social procedure.

The method can include other features described herein.

In another aspect, the invention features a method that includes: obtaining DNA samples from and information about each individual of a first group of the individuals are associated with a trait; analyzing the DNA samples to determine genetic information about a plurality of genetic loci for each individual of the plurality; sending the allelic information to a server that stores genetic information for each individual of a second group of individuals; and receiving information about a subset of individuals selected from the second group of individuals, wherein the subset of individuals is selected using a comparison between the genetic information for members of the first group and genetic information for members of the selected subset. The method can include other features described herein.

In still another aspect, the invention features a server that includes: a memory that stores allelic information for a plurality of genetic markers for each individual of a first group of individuals; and software configured to: receive genetic information about a plurality of genetic loci for each individual of a plurality of individuals; select a subset of individuals from the second group using a comparison between genetic information for members of the plurality of individuals and genetic information for members of the selected subset; and communicate information about individuals of the subset. The software can be configured according to other features described herein.

In another aspect, the invention features a (e.g., a machine-based) method that includes: receiving genetic information for the first and second populations of individuals, the information including information about a plurality of genetic markers for each of the individuals; and returning a scalar value that is a function of the marker distribution for the first and second population and the degree of covariance among the markers. The method can be used, e.g., for comparing a first and second population of individuals. For example, the function is a distance function. The distance can be weighted, e.g., for genetic (e.g., allelic) variability, variance, and co-variance. The distance can be a function of a Euclidean distance, z-score distance, Bhattacharya distance, Mahalanobis distance, Matusita distance, divergence metric, Chemoff distance, angular metric, Earth Mover's distance, Hausdorff distance, City Block (Manhattan) distance, Chebychev distance, Minkowski distance, or Canberra distance. In another example the comparative function is a function of a statistical test, e.g., the mean chi-square of the G-test or a one minus Pearson correlation.

In one example, the function weights each allele by the degree of variability of the respective allele, e.g., by its allele frequency in a third population or the first or second population. The method can include other features described herein.

In another aspect, the invention features a method that includes: receiving information for each individual of a first group of individuals and each individual of a second group of individuals, wherein the information for each individual includes indication about a plurality of different biological features; and evaluating a comparative function that returns a scalar value, compares the information for the first group to information of the second group, and depends on a covariance matrix for at least some features of the plurality of different features. The method can include other features described herein.

In another aspect, the invention features a method that includes: receiving genetic information for a plurality of individuals; identifying a first and second subset of individuals form the plurality of individuals by comparing occurrences of the genetic markers among individuals of the first and second subsets (e.g., complementary, overlapping, or non-complementary subsets); and subjecting the first subset of individuals to a first condition and the second subset of individuals to a second condition. The method can be used, e.g., to perform a controlled study. For example, the first conditions can include administering a test treatment, and the second condition includes administering a control/placebo treatment. In one embodiment, the plurality of individuals includes human individuals consenting to participate in a study.

In another aspect, the invention features a machine readable medium having encoded thereon information including: a first list of records; a second list of records, wherein each record of the first and second list corresponds to a genome and includes genetic information about each of a plurality of genetic loci in the genome; and information describing a relationship between records of the first list and records of the second list, wherein the relationship is a function of the genetic information for at least a subset of the genetic markers, the markers of the subset including markers on at least two different chromosomes, and covariance between alleles of the genetic markers of the subset. The information about the relationship can be stored in a data type that includes a pointer to the first list and a pointer to the second list. For example, the relationship can be based on a result returned by a function or model described herein. For example, the relationship can be a function of distance, e.g., a Mahalanobis distance.

The invention also features algorithms used to implement a comparison described herein and software and systems configured to execute a method described herein. A system can also include a user interface that enables a user to enter, filter, or select information to be used in a comparison and/or to receive a result based on a comparison or information about individuals selected by the system based on a comparison. Instructions for software can be encoded on or in a machine readable or accessible medium. Computer-based methods can be interfaced with a method that includes evaluating a biological sample and generating a computer-interpretable representation about a feature of the biological sample. Computer-based methods can also be interfaced with a user or another computer system, e.g., to provide an interpretable output, e.g., text, graphic, electronic message, sound, or other signal that can be processed by a user. For example, a computer can send identifiers of members of a selected subset to a user or to another computer system.

The term “trait” refers to any detectable property, e.g., a property of an organism, a cell, or a molecule (except a sequence of a genomic DNA). The term “individual” refers to a discrete entity or an item referenced by the discrete entity. For example, in some implementations, an individual can refer to sample obtained from a cell or organism. An “allele” refers to a particular genetic variation in a nucleic acid sequence. Such variation can be present in a gene or outside of a gene. For example, the variation can be present in a coding, non-coding, regulatory, or non-functional region of a nucleic acid sequence. Variations can be present in euchromatin or heterochromatin and so forth.

Methods of the invention can be used, for example, to control for the stratification problem and to correct for type I and type II errors. The methods can be used, for example, to identify a cohort of individuals, or to cluster individuals. Accordingly, methods of the invention can greatly assist the analysis of biological information, for example, genetic analysis and other studies that may be affected by the genetic composition of its subjects. As with all methods pertaining to genetic analysis, methods described herein should accord, in their application, with the highest ethical standards.

Other features and advantages of the instant invention will become more apparent from the following detailed description and claims. Embodiments of the invention can include any combination of features described herein. All patents, patent applications, and publications cited herein are incorporated by reference in their entirety.

### BRIEF DESCRIPTION OF THE DRAWINGS

FIG. 1 depicts haplotype blocks identified in the MTP region of chromosome IV.

FIG. 2 is a flowchart describing an exemplary strategy for identifying a polymorphism associated with longevity.

FIG. 3 is a flowchart of an exemplary method for comparing a case group to a selected control group.

FIG. 4 is a schematic of exemplary data structures.

FIG. 5 is a schematic of an exemplary computer system that can be used to implement aspects of the invention.

### DETAILED DESCRIPTION

In one aspect, the invention features a method for comparing individuals using multiple variables. The method can be used, e.g., to compare one group of individuals to another group and to cluster individuals into groups based on similarities. For example, the method can be used to classify individuals based on a multivariate comparison to a predetermined group of individuals. In one embodiment, the method selects a subset of individuals from a pool based on multivariate comparison to members of the predetermined group to members from a population. The comparison can be used affirmatively to select a subset of individuals that are similar (e.g., most similar) to the predetermined group or it can be used negatively to select a subset of individuals that are dissimilar to the predetermined group. The method is not limited to information about genetic composition and may include information about other characteristics (e.g., in addition to genetic information or instead of genetic information). Application of the method to classify individuals based on genetic compositions is used only as a convenient illustration.

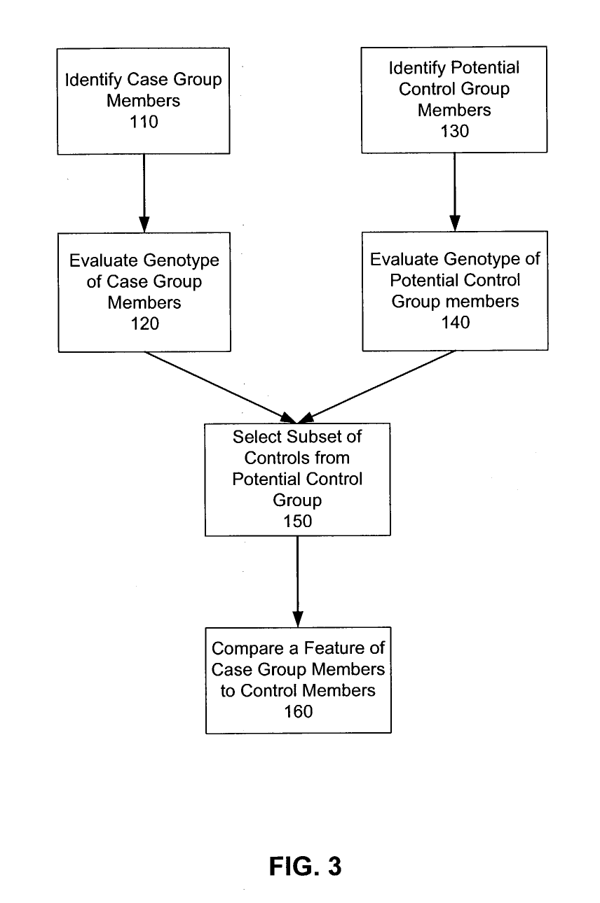

In one implementation, individuals are matched in order to select a control group for another group of individuals (the “case group”). Referring to the exemplary method in FIG. 3, case group members are identified 110. Similarly, potential control group members are identified 130 (e.g., before, after, or concurrently with the case group). The genotype of members of each group are evaluated 120, 140. The genotype can include information about at least one genetic polymorphism. A subset of the potential group members is selected 150. The subgroup is used to define the “control group.” A feature (typically independent of the information used to classify the control group) of members of the case group is compared 160 to members of the control group. For example, statistical methods can be used to evaluate association of a feature with the case group relative to the control group. In one implementation, a LOD score (likelihood of odds) is determined that evaluates the probability that a genetic polymorphism is associated with the case group relative to the control group.

In one example, the case group may be preselected for a particular criterion (e.g., a phenotypic trait). To correlate a genetic polymorphism with a phenotypic trait, the presence of a genetic polymorphism among members of a case group defined by individuals that have the phenotypic trait can be compared to the presence of that polymorphism among members of the selected control group. The LOD score for association between the polymorphism and the trait can be determined. In another example, the case group can be human persons volunteering for an experimental protocol.

In a related aspect, any two groups of individuals are matched. The two groups are identified by a relationship (e.g., a similarity relationship) using a particular model (e.g., a neural network, Bayesian network, or information theory model) or comparison function. The two groups can be distinguished by prior, concurrent, or subsequent criterion. In one example, the two groups can be subjected to separate conditions after the matching. In another example, one group is distinguished by a prior criterion—that is prior to the matching, the first group is selected based on a criterion, and the second group is selected from a general pool based on similarity to the first group. It is possible to use a general pool that has not been evaluated for the criterion. (see, for example, the longevity study below).

Sample matching enables acquisition of statistical information about the association of a feature or multiple features with one or the other groups. Additional groups (e.g., three or more groups) can be identified as needed, e.g., for more complex analyses.

Genetic Information

Genetic information refers to any indication about nucleic acid sequence content. Genetic information can include, for example, an indication about the presence or absence of a particular polymorphism, e.g., one or more nucleotide variations. Exemplary polymorphisms include a single nucleotide polymorphism (SNP), a restriction site or restriction fragment length, an insertion, an inversion, a deletion, a repeat (e.g., trinucleotide repeat, a retroviral repeat), and so forth. In some embodiments, the genetic information describes a haplotype, e.g., a plurality of polymorphisms on the same chromosome. However, in many embodiments, the genetic information is unphased.

It is possible to digitally record or communicate genetic information in a variety of ways. Typical representations include one or more bits, or a text string. For example, a biallelic marker can be described using two bits. In one embodiment, the first bit indicates whether the first allele (e.g., the minor allele) is present, and the second bit indicates whether the other allele (e.g., the major allele) is present. For markers that are multi-allelic, e.g., where greater than two alleles are possible, additional bits can be used as well as other forms of encoding (e.g., binary, hexadecimal text, e.g., ASCII or Unicode, and so forth). The information is typically unphased.

In another embodiment which uses phased genetic information, the first bit is associated with a particular chromosome, e.g., the maternal chromosome, and “0” can be assigned to the minor allele, and “1” can be assigned to the major allele. The second bit is similarly associated with the other chromosome, e.g., the paternal chromosome. In still another embodiment which can be used with unphased genetic information, two bits are used to encode the numbers −1, 0, and 1. Homozygotes for the minor allele were assigned the value −1, heterozygotes 0, and major allele homozygotes 1.

Distance Measures

A distance measure can be used to compare two multivariate variables. The distance is a scalar value that represents a degree of similarity.

One exemplary distance is the Mahalanobis distance. The Mahalanobis distance is a measure of distance between two multivariate means that normalizes each dimension based on the covariance matrix:

D²=(\overline{V}₁−\overline{V}₂)S⁻¹(\overline{V}₁−\overline{V}₂)^{T},

where \overline{V}₁ is a vector representing the mean vector for the cases, \overline{V}₂ is the mean vector for the controls, and S⁻¹ is the inverse of the covariance matrix. The superscript T designates the transform of the difference matrix. D is the Mahalanobis distance. However, for most purposes, D, or any monotonic function of D can be used as an indicator of distance. The member Sᵢⱼ of the covariance matrix S is the covariance between values the i'th and j'th variables, as calculated from pooling data from both the case and control groups. In this matrix, values along the diagonal represent the variance of a particular variable.

Other measures of multivariate distance or similarity that could have been used include Euclidean distance, z-score distance, Bhattacharya distance, Matusita distance, divergence metric, Chernoff distance, angular metric, Earth Mover's distance, Hausdorff distance, one minus Pearson correlation, City Block (Manhattan) distance, Chebychev distance, Minkowski distance, and the Canberra distance. The Euclidian distance, for example, does not account for variance of a particular variable or co-variance between different variables.

Group Selection

There are many ways of selecting a subset of control samples from a set of potential control samples that minimizes a multivariate distance between the case and control groups.

Incremental searching. One example is an incremental search. In one implementation, the single sample that minimizes the distance to the case group is selected from all the potential control samples for inclusion in the control groups. Then, additional samples are added in similar fashion. In other words, in a subsequent cycle, from the remaining potential control samples, the single sample that when added to the previously selected controls sample(s), minimizes the distance to the case group, is selected. In one implementation, the distance is minimized by iteratively calculating the distance between subsets formed by each possible addition and the case group. The subset with the smallest distance is advanced to the next cycle. This step is repeated until the desired number of control samples is selected.

One to one matching. For each sample in the case group, select a sample from the set of potential controls that is most “similar” or “nearest” in multivariate space. The set of one-to-one matched samples are then used as the control group or subjected to other minimization procedures.

Exhaustive search. Another example is an exhaustive search. All possible subgroups (e.g., of a predetermined size or size range) are enumerated and each subgroup is compared to the case group. The subgroup that compares favorably (e.g., most favorably or other favored subgroups) is selected.

Branched searches. This method limits the exhaustive search to a reduced set of possibility. As subgroups are compared, possible combinations are eliminated, e.g., using the dead-end theorem or other branching methods, to enumerate only some subgroups from the universe of possible subgroups.

Preclustering. It is also possible to compare members of the potential controls to one another to identify clusters of similar members using a comparison function. Then clusters that are similar to individual members or clusters in the case group are selected for inclusion in the control group.

Prefiltering. Prefiltering criteria can be defined to reduce the search size. For example, if all members of the case group have a certain properties, it is possible to eliminate members of the potential controls that do not have these properties. For example, if all members of the case group have the same alleles of at particular loci, potential controls that do not have these markers are discarded.

Boundary methods. A distance measure can also be used to define a boundary in multivariate space that defines a range similarity to members of the case group. For example, a Mahalanobis group can be defined using the Mahalanobis distance function. Controls can be selected from the subset of members that are within the boundary.

As described above, matching is evaluating using a distance function for multivariates. However, other methods can be used. For example, a Bayesian network or a model-based on information theory can be used.

The success of the matching can depend on the number of markers used, the informativeness of the markers with respect to genetic background, the similarities between the cases and controls being matched, and the degree of over sampling that occurs. Although described above as a selection of “controls” best matched to “cases”, the opposite works equally as well, and “case” and “control” are only labels to distinguish two groups of samples that are distinguished by some covariate (e.g. trait, phenotype, etc.). Similarly, the comparisons need not be based only on genetic information, but can include, in addition, other biological information, or exclusively non-genetic information.

The matching can be evaluated using a second function, e.g., another distance metric or a statistical function. For matching genetic backgrounds, the mean chi-square of the G-Test statistics can be used to evaluate the matching. If the genetic backgrounds of the two armed study were perfectly matched, the mean chi-square of the G-Test statistics for these markers have an expected value of 1.0. In some embodiments, a threshold may be set for the mean chi-square of the G-Test statistics, e.g., less than 1.4, 1.3, 1.2, 1.1., or 1.0.

Exemplary Applications

In one embodiment, the method can be used to identify two cohorts of genomes that are balanced relative to each other. The genomes can be from individual organisms, cells, and so forth. One application is to identify a control group of individuals for an experimental (or test) group, particularly where matching the genetic backgrounds of the two groups is important for evaluating data from the experimental and control groups.

The method can be used to identify a control group of individuals that is balanced relative to a test group. For example, the method can be used to evenly match individuals in test and control groups. The method can be used to partition individuals into two groups balanced for a plurality of biological parameters, e.g., genetic composition and/or other biological parameters described herein. Balancing can be general or targeted. General balancing typically involves, e.g., selecting genetic markers without regard for their chromosomal position or association with particular traits. For example, these genetic markers may be distributed randomly throughout the genome, e.g., on at least two chromosomes. General balancing can be used to optimize the genetic backgrounds of the test and control groups. In contrast, targeted balancing can be used to optimize the distribution of heterogeneity in one or more specific regions of the genome between the test and control groups. For example, in a study of a treatment for Alzheimer's disease, it may be useful to if the test and control groups include similar distributions of alleles known to be associated with that disease.

It is also possible to select genetic markers based on certain criteria, e.g., criteria that are independent of map position. Exemplary criteria include criteria that depend on distribution of the marker in a population, e.g., a sample population. Such criteria include: the relative prevalence of the major and minor allele, and degree of heterozygosity (e.g., between 0.1-5%, 3-20%, 20-45%, or 30-50%. Exemplary criteria can also include experimental factors, e.g., degree of certainty that the allele can unambiguously be identified. Other criteria may include: reliability of assay with respect to a specific platform and informativeness of a marker with respect to the genetic background of individuals sampled.

It is possible to survey a broad class of individuals that can qualify as potential controls and identify a panel of biological markers (e.g., genetic markers) that vary among the potential controls. The panel of markers can then be used to select the subset of controls by comparison to the case group. If required, variance and/or covariance is used as a component of the comparison function to control for the degree of variation.

In some embodiments, the genetic markers are selected based on map position, e.g., distance from another marker, distance from a centromere or telomere, and distance from heterochromatin.

The methods can be used to map genes that affect a trait of any organism, particularly a polyploid (e.g., diploid) sexual organism. For example, the method can be used to map genes that may be associated with a human disease, and other human traits, such as resistance to environmental conditions, physical manifestations, and behaviors. In just one application, the method is used to evaluate genes that affect lifespan regulation or an age-related disease or predisposition to such a disease. Exemplary age-related diseases include: cancer (e.g., breast cancer, colorectal cancer, CCL, CML, prostate cancer); skeletal muscle atrophy; adult-onset diabetes; diabetic nephropathy, neuropathy (e.g., sensory neuropathy, autonomic neuropathy, motor neuropathy, retinopathy); obesity; bone resorption; age-related macular degeneration, AIDS related dementia, ALS, Alzheimer's, Bell's Palsy, atherosclerosis, cardiac diseases (e.g., cardiac dysrhythmias, chronic congestive heart failure, ischemic stroke, coronary artery disease and cardiomyopathy), chronic renal failure, type 2 diabetes, ulceration, cataract, presbiopia, glomerulonephritis, Guillan-Barre syndrome, hemorrhagic stroke, rheumatoid arthritis, inflammatory bowel disease, multiple sclerosis, SLE, Crohn's disease, osteoarthritis, Parkinson's disease, pneumonia, and urinary incontinence. Symptoms and diagnosis of such diseases are well known to medical practitioners.

Similarly, the method can be used to map genes that affect traits of other animals, e.g., agricultural livestock and wild animals. Further, the method can be used to map genes of plants, and sexual parasites.

In another embodiment, the method can be used to identify two cohorts of individuals that are balanced relative to each other based on biological parameters, e.g., molecular parameters, levels of metabolites, gene expression, protein modification and so forth. The parameters can be evaluated by analyzing individual organisms, organs, tissues, cells, and so forth. One application is to identify a control group of individuals for an experimental (or test) group, particularly where matching the biological state of the two groups is important for evaluating data from the experimental and control groups.

Methods of Evaluating Genetic Information

There are numerous ways of evaluating genetic information. Nucleic acid samples can analyzed using biophysical techniques (e.g., hybridization, electrophoresis, and so forth), sequencing, enzyme-based techniques, and combinations-thereof. For example, hybridization of sample nucleic acids to nucleic acid microarrays can be used to evaluate sequences in an mRNA population and to evaluate genetic polymorphisms. Other hybridization based techniques include sequence specific primer binding (e.g., PCR or LCR); fluorescent probe based techniques Beaudet et al. (2001) Genome Res. 11(4):600-8. Electrophoretic techniques include capillary electrophoresis and Single-Strand Conformation Polymorphism (SSCP) detection (see, e.g., Myers et al. (1985) Nature 313:495-8 and Ganguly (2002) Hum Mutat. 19(4):334-42).

In one embodiment, allele specific amplification technology that depends on selective PCR amplification may be used to obtain genetic information. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it is possible to introduce a restriction site in the region of the mutation to create cleavage-based detection (Gasparini et al. (1992) Mol. Cell Probes 6:1). In another embodiment, amplification can be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Enzymatic methods for detecting sequences include amplification based-methods such as the polymerase chain reaction (PCR; Saiki, et al. (1985) Science 230, 1350-1354) and ligase chain reaction (LCR; Wu. et al. (1989) Genomics 4, 560-569; Barringer et al. (1990), Gene 1989, 117-122; F. Barany. 1991, Proc. Natl. Acad. Sci. USA 1988, 189-193); transcription-based methods utilize RNA synthesis by RNA polymerases to amplify nucleic acid (U.S. Pat. No. 6,066,457; U.S. Pat. No. 6,132,997; U.S. Pat. No. 5,716,785; Sarkar et al., Science (1989) 244:331-34; Stofler et al., Science (1988) 239:491); NASBA (U.S. Pat. Nos. 5,130,238; 5,409,818; and 5,554,517); rolling circle amplification (RCA; U.S. Pat. Nos. 5,854,033 and 6,143,495) and strand displacement amplification (SDA; U.S. Pat. Nos. 5,455,166 and 5,624,825). Amplification methods can be used in combination with other techniques.

Mass spectroscopy (e.g., MALDI-TOF mass spectroscopy) can be used to detect nucleic acid polymorphisms. In one embodiment, (e.g., the MassEXTEND™ assay, SEQUENOM, Inc.), selected nucleotide mixtures, missing at least one dNTP and including a single ddNTP is used to extend a primer that hybridizes near a polymorphism. The nucleotide mixture is selected so that the extension products between the different polymorphisms at the site create the greatest difference in molecular size. The extension reaction is placed on a plate for mass spectroscopy analysis.

Fluorescence based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (i.e., “color”) of the fluorescent dye that is incorporated.

Hybridization to microarrays can also be used to detect polymorphisms, including SNPs. For example, a set of different oligonucleotides, with the polymorphic nucleotide at varying positions with the oligonucleotides can be positioned on a nucleic acid array. The extent of hybridization as a function of position and hybridization to oligonucleotides specific for the other allele can be used to determine whether a particular polymorphism is present. See, e.g., U.S. Pat. No. 6,066,454.

It is also possible to directly sequence the nucleic acid for a particular genetic locus, e.g., by amplification and sequencing, or amplification, cloning and sequence. High throughput automated (e.g., capillary or microchip based) sequencing apparati can be used.

Any combination of the above methods can also be used.

Other Methods of Evaluating Biological Parameters

Other molecular, genetic, cellular, immunological, and other biological methods known in the art can also be used to evaluate a property of a biological system. For general guidance, see, e.g., techniques described in Sambrook & Russell, Molecular Cloning: A Laboratory Manual, 3^{rd} Edition, Cold Spring Harbor Laboratory, N.Y. (2001), Ausubel et al., Current Protocols in Molecular Biology (Greene Publishing Associates and Wiley Interscience, N.Y. (1989), (Harlow, E. and Lane, D. (1988) Antibodies: A Laboratory Manual, Cold Spring Harbor Laboratory Press, Cold Spring Harbor, N.Y.), and updated editions thereof.

For example, antibodies, other immunoglobulins, and other specific binding ligands can be used to detect biomolecule, e.g., a protein or other antigen. For example, one or more specific antibodies can be used to probe a sample. Various formats are possible, e.g., ELISAs, fluorescence-based assays, Western blots, and protein arrays. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1.

Proteins can also be analyzed using mass spectroscopy, chromatography, electrophoresis, enzyme interaction or using probes that detect post-translational modification (e.g., a phosphorylation, ubiquitination, glycosylation, methylation, or acetylation).

Nucleic acid expression can be detected, e.g., for one or more genes by hybridization based techniques, e.g., Northern analysis, RT-PCR, SAGE, and nucleic acid arrays. Nucleic acid arrays are useful for profiling multiple mRNA species in a sample. A nucleic acid array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

Metabolites can be detected by a variety of means, including enzyme-coupled assays, using labeled precursors, and nuclear magnetic resonance (NMR). For example, NMR can be used to determine the relative concentrations of phosphate-based compounds in a sample, e.g., creatine levels. Other metabolic parameters such as redox state, ion concentration (e.g., Ca²⁺)(e.g., using ion-sensitive dyes), and membrane potential can also be detected (e.g., using patch-clamp technology).

Imaging techniques (including NMR, tomographic, radiological, and microscopic methods) can be used to image a sample or an organism. Examples of imaging information include the localization (e.g., tissue or sub-cellular) of a biomolecule (e.g., a protein, mRNA, or metabolite). Some imaging techniques use probes, e.g., probes such as fluorescent labels such as fluorescein and rhodamine, nuclear magnetic resonance active labels, Short-range radiation emitters, positron emitting isotopes detectable by a positron emission tomography (“PET”) scanner, chemiluminescers such as luciferin, and enzymatic markers such as peroxidase or phosphatase.

Fluorescence activated cell sorting can be used to profile a cell population (e.g., blood cells). FACS analysis can use one or more labeled antibodies for typing cells, e.g., using cell surface markers. Cells can also be assayed for response to a stimulus, e.g., to a signalling molecule or other perturbation.

Numerous other assays can be used to detect the presence, quality, or quantity of a biomolecule or other biological property. Whole organisms can be assayed, e.g., by exposure to a pathogen, for a behavioral response, and so forth.

Computer Implementations

The invention can be implemented in digital electronic circuitry, or in computer hardware, firmware, software, or in combinations thereof. Methods of the invention can be implemented using a computer program product tangibly embodied in a machine-readable storage device for execution by a programmable processor; and method actions can be performed by a programmable processor executing a program of instructions to perform functions of the invention by operating on input data and generating output. For example, the invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language. Suitable processors include, by way of example, both general and special purpose microprocessors. A processor can receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks. Any of the foregoing can be supplemented by, or incorporated in, ASICs (application-specific integrated circuits).

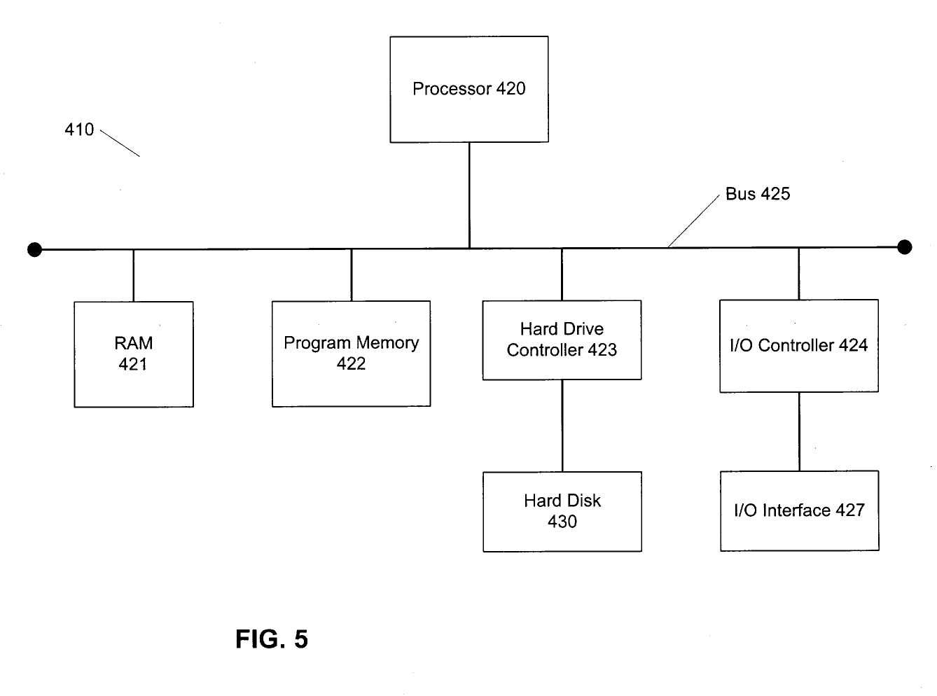

An example of one such type of computer is depicted in FIG. 5, which shows a block diagram of a programmable processing system (system) 410 suitable for implementing or performing the apparatus or methods of the invention. The system 410 includes a processor 420, a random access memory (RAM) 421, a program memory 422 (for example, a writable read-only memory (ROM) such as a flash ROM), a hard drive controller 423, and an input/output (I/O) controller 424 coupled by a processor (CPU) bus 425. The system 410 can be preprogrammed, in ROM, for example, or it can be programmed (and reprogrammed) by loading a program from another source (for example, from a floppy disk, a CD-ROM, or another computer).

The hard drive controller 423 is coupled to a hard disk 430 suitable for storing executable computer programs, including programs embodying the present invention, and data including storage. The I/O controller 424 is coupled by means of an I/O bus 426 to an I/O interface 427. The I/O interface 427 receives and transmits data in analog or digital form over communication links such as a serial link, local area network, wireless link, and parallel link.

One non-limiting example of an execution environment includes computers running Linux Red Hat OS, Windows NT 4.0 (Microsoft) or better or Solaris 2.6 or better (Sun Microsystems) operating systems. Browsers can be Microsoft Internet Explorer version 4.0 or greater or Netscape Navigator or Communicator version 4.0 or greater. Computers for databases and administration servers can include Windows NT 4.0 with a 400 MHz Pentium II (Intel) processor or equivalent using 256 MB memory and 9 GB SCSI drive. For example, a Solaris 2.6 Ultra 10 (400 Mhz) with 256 MB memory and 9 GB SCSI drive can be used. Other environments can also be used.

In one implementation, information about a set of potential controls is stored on a server. A user can send information about case groups to the server, e.g., from a remote computer that communicates with the server using a network, e.g., the Internet. The server can compare the information about the case groups and select a subset of members from the potential controls, e.g., to minimize a distance measure that is a function of the case groups and the selected subset. The server can return information about the subset (e.g., identifiers or other data) to the user or can return an evaluation that compares a feature of the case group to the members of the selected subset (e.g., a statistical score that evaluates probability of association with the case group relative to the selected subset). Accordingly, the server can include a electronic interface for receiving information from a user or from an apparatus that provides information about a biological property and software configured to execute identify a subset of data objects using a comparison described herein.

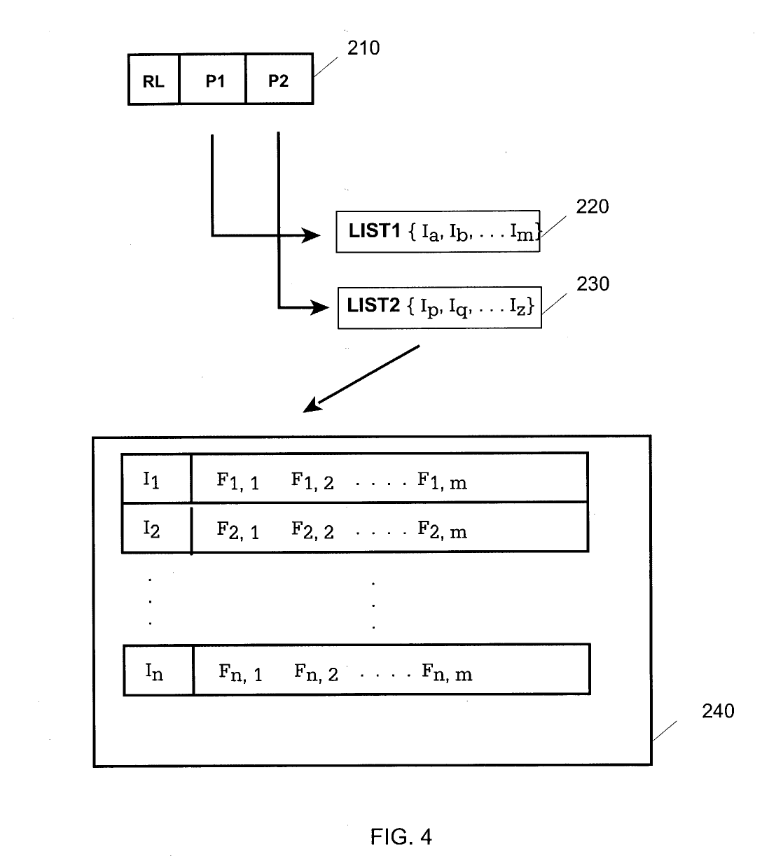

Referring to the exemplary data structures in FIG. 4, the server can store a data type 210 which includes information (RL) that relates two sets of the individuals and a table 240 which includes information about the individuals (indexed by I₁, I₂, . . . Iₙ). For example, the information about the first individual in the table 240 can include an index I₁, and features (F_{1,1}, F_{1,2}, and so on to F_{1,m}). The features can be, e.g., the presence of a genetic polymorphism. The data type 210 includes a first pointer (P1) and a second pointer (P2). P1 references a list 220 of individuals by their index in the table 240. P2 references another list 230 of individuals in the table 240. Other methods of referencing the individuals (e.g., without an index) can also be used. The field RL in the datatype 210 can be used to store information about how the first list 220 relates to the second list 230. For example, RL can be used to store a scalar distance value or a vectorial value that is the result of a comparison function or a model that compares the two members of the two lists.

In some implementations, it is possible to include a table (not shown) that stores the data type 210 in each row, and optionally additional fields. Such a table can be used during a procedure that searches for a favored set of related groups. Thus, a relational database of the invention can include three tables, the table 240, a table that includes the data type 210, and a table of lists 220 and 230.

The following non-limiting example illustrates a particular implementation of sample matching.

### EXAMPLE

In a genome-wide linkage study for human longevity using 308 long-lived individuals (centenarians or near-centenarians) in 137 sibships, a locus was identified with statistically significant linkage within chromosome IV near microsatellite D4S1564. This interval spans 12 million base pairs and contains approximately 50 putative genes. A haplotype-based fine mapping was used to study the interval and identify the specific gene and gene variants impacting lifespan. The resulting genetic association study identified a single gene, microsomal transfer protein (MTP) accounting for significant variance in human lifespan. MTP has been identified as the rate limiting step in lipoprotein synthesis and may affect longevity by subtly modulating this pathway. This study provides proof of concept for the feasibility of fine mapping linkage peaks using association studies and for the power of using the centenarian genome to identify genes impacting longevity.

The ability to survive to old age is partially under genetic influence (McGue, Vaupel et al. 1993; Herskind, McGue et al. 1996; Gudmundsson, Gudbjartsson et al. 2000; Perls, Shea-Drinkwater et al. 2000). In the most intuitive cases, individuals burdened by the fatal monogenic diseases of youth, such as cystic fibrosis, retinoblastoma, and muscular dystrophy have a reduced lifespan compared with the general population. However, although the effects of these harmful gene variants is large in magnitude with respect to affected individuals, because these mutations are extremely rare, all the monogenic diseases combined contribute little to the population variance in human lifespan.

There is demographic evidence that there is considerable heritability of human lifespan. Based on an analysis of longevity in twins, this heritability has been estimated at 25%, however the importance of genetic factors is likely greater at the extremes of age. For example, male and female siblings of centenarians have 17-fold and 8-fold greater relative risks respectively of surviving to age 100 and about half the death rate from age 20 to age 100 of birth-cohort matched individuals (Perls, Wilmoth et al. 2002).

These studies suggest that exceptional longevity is amenable to genetic studies, but not without the realization that achieving one hundred years represents a complex interaction of genetics, environment, and chance. Lifespan can be conceptualized as the most complex trait of all, as this trait necessarily integrates genetic and environmental factors contributing to all diseases affecting human mortality. Accordingly, genetic variance in human lifespan within a population may be distributed over many genes with relatively subtle influences by any single gene. The distribution of these effects (e.g. the number of genes accounting for much of the genetic variance) is an unanswered empirical question. If the variance in human lifespan is evenly distributed over large numbers of genes and gene variants (alleles), the likelihood of deciphering the individual contributions is small. Furthermore, if unspecified gene-gene and gene-environment interactions account for the majority of the variance, these difficulties will be compounded. Despite these concerns, an increasing number of genetic studies are reporting genes associated with human longevity. These genes include ApoE, ApoB, and klotho (Kervinen, Savolainen et al. 1994; Schachter, Faure-Delanef et al. 1994; van Bockxmeer 1994; Arking, Krebsova et al. 2002), although only ApoE has been reproduced consistently. In order to achieve their extreme age, centenarians likely lack numerous gene variants that are associated with premature mortality and there is also the possibility that they are more likely to carry protective variants as well (Wachter 1997; Schachter 1998).

From Linkage Study to Association Study

Results of a genome-wide linkage scan using 308 extremely long lived individuals in 137 sibships and linkage to exceptional longevity (i.e. living beyond the 5% survival tail) at chromosome IV near microsatellite D4S1564 with a maximum LOD score of 3.65 (p=0.044 genome-wide with non-parametric analysis) have been reported (Puca, Daly et al. 2001). No other chromosomal region achieved statistically significant linkage in this study. There are approximately 50 putative genes in the 12 million base pairs spanning the 85% confidence interval of this linkage peak, and a priori it was difficult to exclude any of the genes based on functional considerations. In addition, it was possible that the polymorphism underlying the linkage was not within any of these 50 “genes.” Therefore, an unbiased, systematic fine mapping of the region was desired. Although it would be important to identify the specific polymorphisms involved, this was not possible with the resolution provided by family-based linkage studies, compounded with the difficulty of collecting larger numbers of sibling-pairs.

This study finely mapped the chromosome IV locus with the hope of identifying specific gene variants associated with exceptional longevity. Rather than bias the potential findings to regions of the locus containing well characterized genes, a systematic exploration of the linkage peak was conducted. With this aim, 2,000 single nucleotide polymorphisms (SNPs) (an average of one every 6 kb) within the longevity linkage locus were selected from the SNP consortium (TSC) database. Based on experience with an earlier pilot study, only a fraction of these markers were expected to be useful in an association study. Of the 2000, a total of 875 SNPs were converted into successful genotyping assays and were determined to be polymorphisms with minor allele frequency greater than 5%.

From SNPs to Haplotypes

Although these validated SNP assays could have been used alone as markers in the association study described below, there were strong arguments to additionally build a haplotype map of the locus from these SNPs and then leverage the reconstructed haplotypes as genetic markers. A haplotype is a specific combination of alleles of nearby markers. In most cases, the power (informativeness) of a genetic marker with respect to an association study is increased when there are large numbers of variants of a single marker (unless the marker is the causative variant). Accordingly, SNP markers, which are biallelic, have less power to detect associations than multi-SNP haplotypes. Secondly, the diversity of the genome can be effectively captured by reducing it to sequential blocks of haplotypes with limited diversity (Johnson, Esposito et al. 2001; Patil, Berno et al. 2001; Stephens, Schneider et al. 2001). Defining haplotypes provides the opportunity for selecting groups of markers which are minimally correlated with one another, which maximizes the statistical power per marker. Once the common haplotypes within a block have been defined, SNPs within the same block redundant for discriminating between the different haplotypes can be omitted for defining haplotypes. After removing SNPs redundant with respect to defining haplotypes within each block, 875 validated SNPs and approximately 700 “maximally informative SNPs” remained for using in association studies (see supplementary information). Finally, haplotype reconstruction provides a number of ways to assess the statistical coverage of a mapping effort and to model the recombination history within a locus.

Haplotype based approaches applied to smaller genomic regions have been demonstrated by others (Daly, Rioux et al. 2001; Johnson, Esposito et al. 2001; Rioux, Daly et al. 2001) and advantages over single markers have been shown (De Benedictis, Falcone et al. 1997; Stephens 1999; Davidson 2000). There is no generally accepted method for defining and recovering haplotypes from SNP-based data. The algorithms used in this study are outlined in the Methods.

Testing for Association

By densely genotyping across the 12 Mb region, a good draft of the underlying haplotype structure was constructed. Approximately 75% of the mapped region was within regions of strong linkage disequilibrium. Using this carefully reconstructed assortment of SNP-based haplotype markers, a case/control association study between groups of unrelated long-lived individuals (age 98 and older) and a much younger control population (less than 50 years of age) was conducted.

To reduce genotyping costs and to increase the power by confirming the hypothesis in independent populations, the study was divided in two sequential tiers of samples, with the first tier comparing 190 centenarians with 190 controls at SNP-based haplotype markers. These initial sample sizes were intended only as a preliminary screen of the region. This first attempt pointed in the direction of the MTP gene. Although several SNPs and haplotype markers were “significant” at p<0.05, the marker showing the strongest association (p=0.0005) was the SNP rs1553432, located 72 kb upstream from MTP. This association provided a potentially interesting first hypothesis to follow up with dense genotyping and haplotype mapping of the surrounding genes. A review of the December 2001 human genome draft showed four nearby areas of interest—the alcohol dehydrogenase (ADH) gene cluster, the partially characterized transcripts AL136838, AK000332 and microsomal transfer protein (MTP).

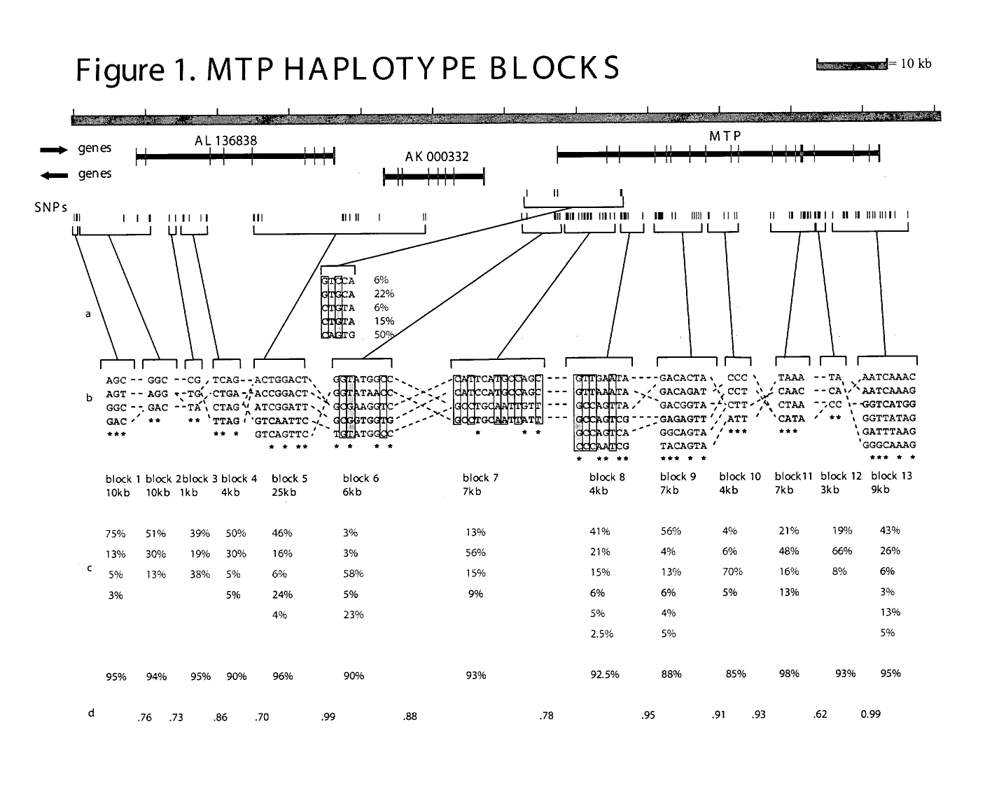

In the 250 kb region bracketing rs1553432, 60 SNPs were identified and validated. Several of these densely spaced SNPs showed strong associations when analyzed in the set of 190 cases and controls used above; most of these markers were located near the 5′ end of MTP or just upstream of this gene, particularly densely near the promoter. All of the newly identified associations were in strong linkage disequilibrium with rs1553432 (e.g., they fell on the same “long-range” haplotype). With interest narrowing in on a single gene, all known SNP polymorphisms for MTP and its promoter were genotyped in the original 190 cases (long lived individuals) and 190 controls (young individuals). After haplotype reconstruction of the area was completed, a single haplotype (see FIG. 1a), which was underrepresented in the long-lived individuals, accounted for the majority of the statistical distortion at the locus. Genotyping an additional 190 cases and controls further increased the strength of the association at this locus (p=0.000005, relative risk=0.56). See Table 1 for counts and frequencies of the haplotypes compared. This haplotype was seen in 27% of controls and 17% of long-lived individuals. Two of the many SNPs within this block (rs2866164 and MTP Q/H 95) were sufficient to distinguish this allele from all others. These two SNPs were interesting because of their potential functional significance. RS2866164 is perfectly correlated with another MTP promoter SNP, rs1800591 (also known as −493 G/T) that has been previously associated with several phenotypes including lipoprotein profiles, central obesity, and insulin resistance (see below). MTP Q/H 95, although not known to have any functional significance, results in a semi-conservative amino acid change (from glutamine to histidine) in exon three at the protein's 95th translated amino acid.

TABLE 1
Risk allele frequencies

| | |
|---|---|
| −493 G allele | −493T allele |

Cases (long-lived)

| | | |
|---|---|---|
| 95 Q allele | 546 (76%) | 127 (17%) |
| 95H allele | 0 | 53 (7%) |

Controls

| | | |
|---|---|---|
| 95 Q allele | 498 (68%) | 201 (27%) |
| 95H allele | 0 | 36 (5%) |

Table 1. Risk haplotype allele frequencies. Broken down into cases (long-lived) and controls, shows frequencies for the four possible haplotypes defined by the promoter (−493 G/T) and exon 3 (95 Q/H) polymorphisms. Note that only three of the four haplotypes was observed, fulfilling the criteria of no historic recombination between the two SNPs. 726 out of 760 case chromosomes were successfully genotyped at both alleles in the long-lived individuals, compared to 735 out of 760 for the controls. As discussed in the text, the haplotype composed of the −493T allele and 95Q allele is underrepresented in long-lived individuals, suggesting this variant confers mortality risk. Note that the MTP −493 marker has multiple “twins” displaying identical statistical behaviour (see text).

Genetic Stratification and Controlling Type I Error

Some genetic association studies have been plagued with false positive or other problematic results (Hirschhorn, Lohmueller et al. 2002). A recognized problem affecting genetic association studies is a failure to adequately match the genetic backgrounds of the cases and controls, a phenomenon called stratification. This association study which compares individuals born decades apart can be potentially vulnerable to this confounder because the geographic distribution of ethnicities has changed over the past 100 years. Specifically, this case population reflects the ethnic distribution of the United States near the beginning of the last century while the control population was sampled from more recent generations. To minimize this problem, only DNA from people who identified themselves as “Caucasian” was used but even this class is obviously a diverse group.

Consequently, cases and controls would differ not only with respect to the longevity phenotype but also have ethnicity as an uncontrolled confounder. If the effect is strong enough, associations will be found reflecting these ethnic differences rather than differences in lifespan. There are accepted ways of checking and correcting for potential stratification, one of which is described in the Methods. The mean chi-square for randomly selected SNP markers (representing differences in genetic background) for the 380 cases and controls tested above was 1.51 (compared with an expected value of 1.0). Although, modest, any amount of stratification is undesirable and the methods of correcting for this potential confounder have not been empirically well validated.

To avoid correcting for the hundreds of partially independent hypotheses tested with the original sample set and to simultaneously eliminate stratification as a problem, proactive sample matching was used. 250 cases were proactively matched (see also below) against individuals selected from a new set of 463 potential controls. Using the approach discussed in the Methods section, a subgroup of 250 controls from the potential controls was selected that best matched the cases with respect to genetic background. The mean chi-square for this group of samples (using and independent group of SNPs) was 0.92, indicating a very high level of genetic balance. None of these samples was used to generate the single hypothesis being pursued, allowing testing the single inference that the risk haplotype was underrepresented in long-lived individuals. The association at this haplotype was confirmed with this well matched group of cases and controls (p=0.01 by G-Test, p=0.0027 by Hotelling-T test, relative risk=0.69).

Although the interaction between rs2866164 and Q/H 95 was sufficient to account for all the association at the locus, it is imprudent to conclude that the polymorphisms were causative with respect to longevity. In particular, a few “twins” (SNPs whose alleles are perfectly correlated) of −493 G/T were identified that, in combination with Q/H 95 could equally explain the data. Ideally, because simpler models are preferred over more complex solutions, a single SNP “tagging” (i.e. distinguishing) the risk haplotype would be favored over the two SNP interaction model.

To search for “tagging” SNPs, a resequencing strategy intended to minimize the number of samples assayed was used. The details of this strategy are described in the Methods. This procedure was applied to the 12 kb within the risk block and the 72 kb block of DNA extending up to the initial rs1553432 SNP. In addition, all 18 exons of MTP were sequenced in a group of 50 long-lived individuals to search for rare functional polymorphisms that would not fall on well-defined haplotypes. Altogether, 104 SNPs were identified, although none uniquely tagged the risk haplotype. After adding the additional SNPs to the map, a new block structure was defined with significant changes (FIG. 1b), but no evidence of recombination between MTP −493 G/T and MTP Q/H 95 was observed. Because a single SNP marker could not explain the association, the most parsimonious model involved an interaction between the two original functional SNPs.

After confirming the MTP finding, there remained the possibility that an additional gene associated with longevity could be contributing to the linkage peak. To be as thorough as possible, all of the hundreds of SNPs or SNP-based haplotypes genotyped in the first set of 190 cases and controls significantly associated at p<0.05 was tested in independent samples, as described above for MTP. At the end of this sequential process, there were no additional associations that survived the proper corrections discussed above, although larger sample sizes and/or more perfect sample matching may reveal additional associations in the future. 190 cases and controls were genotyped using at least 5 SNPs near all the well-characterized genes under the locus, which involved assaying an additional 55 SNP markers. This effort yielded no additional associations, leaving MTP as the lone candidate to explain the original linkage result.

MTP Biology and Previous Associations

The gene product of MTP has been well characterized since the mid 1980s for its role in lipoprotein assembly and is an investigational target for treating combined hyperlipidemia and obesity (Wetterau, Lin et al. 1997; Shelness and Sellers 2001). MTP is thought to be the rate limiting step in production of apoB containing particles (Jamil, Chu et al. 1998), making it a particularly appealing target for next-generation lipid-lowering drugs. Structurally, the protein dimerizes with the ubiquitous protein disulfide isomerase (PDI) and resides on the luminal surface of the endoplasmic reticulum (ER) where it facilitates the proper manufacturer of very low density lipoprotein (VLDL) and chylomicron particles. Functionally, MTP is directly involved in the packaging of apoB and triglyceride into these particles, and MTP and apoB are thought to directly bind one another during this assembly (Wu, Zhou et al. 1996). Rare humans with two non-functioning copies of the gene suffer from abetalipoproteinemia, and are characterized by the near absence of Apo-B particles in serum (Berriot-Varoqueaux, Aggerbeck et al. 2000). To survive, these individuals must be aggressively treated with fat soluble vitamin supplementation.

MTP has been studied in animal models. The single copy knockout of MTP in mice resulted in a 28% reduction in ApoB levels while homozygotes died during embryonic development (Raabe, Flynn et al. 1998). Hepatic overexpression in transgenic mice results in increased in vivo secretion of VLDL and apoB (Tietge, Bakillah et al. 1999). A liver-specific double knockout in mice lowered apoB-100 levels by 95% and apoB-48 levels by only 20% (Raabe, Veniant et al. 1999). Liver specific single copy MTP knockout mice demonstrate reduced serum glucose, insulin, and triglyceride levels, suggesting the additional importance of this gene in metabolic disease (Bjorkegren, Beigneux et al. 2002). Numerous classes of drugs that inhibit MTP activity have been shown to improve lipoprotein profiles (Wetterau, Gregg et al. 1998). Several food-products have also been shown to reduce MTP activity, including garlic (Lin, Wang et al. 2002), ethanol (Lin, Li et al. 1997), and citric flavanoids (Wilcox, Borradaile et al. 2001). One study found that MTP promoter allele −493T up-regulated MTP expression by two-fold (Karpe, Lundahl et al. 1998).

MTP has been associated with phenotypes including lipoprotein profiles, insulin resistance, and fat distribution, and most of these studies focused on the −493G/T marker (Herrmann, Poirier et al. 1998; Karpe, Lundahl et al. 1998; Couture, Otvos et al. 2000; Juo, Han et al. 2000; Talmud, Palmen et al. 2000; Ledmyr, Karpe et al. 2002; St-Pierre, Lemieux et al. 2002). In terms of linkage studies, one investigation uncovered a quantitative trait locus (QTL) for lipoprotein particle size that included the MTP gene (Rainwater, Almasy et al. 1999). A linkage study of dizygotous twins implicated MTP in regulating triglyceride levels, which have been tentatively identified as a coronary artery disease modulator (Austin, Talmud et al. 1998). Like some other mapping studies of complex traits, the literature surrounding MTP has been complex and often contradictory. Genetic stratification and a failure to consider the 95 Q/H polymorphism may have contributed to the confusion. Given the inconsistent phenotype associations attributed to this gene in the past, it will be important to confirm the longevity association in independently ascertained collections.

The known activity of MTP, as a rate limiting step in lipid metabolism, is consistent with a relationship between MTP and human longevity. Coronary artery disease and other vasculopathies attributed to unfavorable lipid profiles (peripheral vascular disease, renal-vascular disease, and stroke) account for a large percentage of human mortality. Common genetic variants that impact the function of lipid metabolism should be expected to impact human lifespan; for example the offspring of centenarians have higher levels of HDL (good cholesterol) and lower levels of LDL (bad cholesterol) than age matched controls and they demonstrate significantly lower risks of heart disease and stroke compared with age-matched controls (Barzilai, Gabriely et al. 2001; Terry, Wilcox et al. 2003). In addition, a “longevity syndrome” was described amongst families with extremely low levels of LDL particles (Glueck, Gartside et al. 1977). Although reasonable to believe that the impact of MTP on human longevity is through its impact on lipid profiles, the association studies above suggest that this gene may also affect susceptibility to insulin resistance and obesity.

MTP and APOE

There are many parallels between the associations of MTP and APOE. Both genes are risk factors implicated in cardiovascular disease as well as longevity, and the latter being also associated with Alzheimer's. The genetic epidemiology of MTP can be compared to incidence and predisposition of age-related diseases, such as Alzheimer's. Before starting the current study, as a quasi positive control, it was confirmed that in the subject population that the apo-E ε2 allele is protective, the ε3 allele is neutral, and the ε4 allele is detrimental with respect to lifespan extension. No interaction between the MTP and APOE alleles with respect to lifespan was detected, although sample size may have been inadequate.

Some Implications

This study demonstrates that centenarians and near-centenarians can serve as a model for studying human longevity and disease resistance (Barzilai, Gabriely et al. 2001). A population that has escaped or delayed the lethal pathologies of old age is useful for detecting genetic factors that impact the diseases of aging (Silverman, Smith et al. 1999). Here, a haplotype-based linkage disequilibrium mapping approach identified a risk allele based on an initial finding contributed by a linkage study. The complex trait linkage peak ultimately resulted in the identification of a specific gene variant.

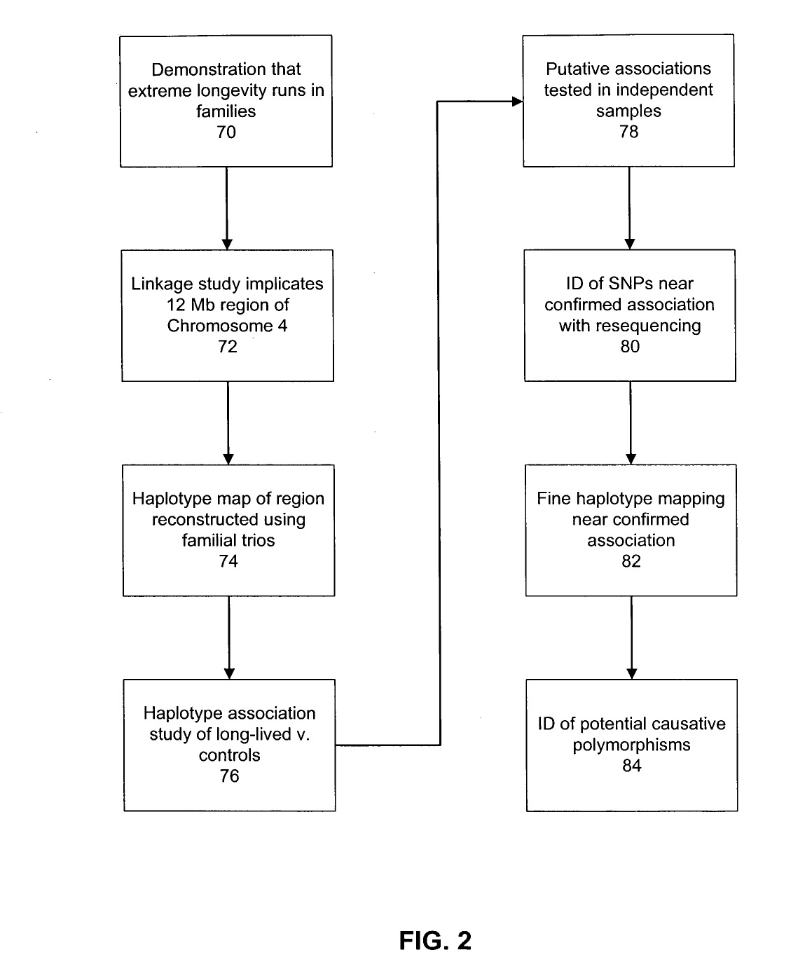

FIG. 1 depicts haplotype-blocks at MTP locus: (a) The original haplotype block defined by publicly available SNPs containing RS2866164 (circled box) and MTP Q/H 95 (boxed). The arrow indicates the risk haplotype. (b) a more refined map that include 61 novel SNPs showing MTP −493 G/T (boxed) and MTP Q/H 95 belonging to different haplotype blocks but in strong linkage disequilibrium. In circled boxes there are SNPs perfectly correlated with MTP −493 G/T. Dashes lines indicate haplotypes which are commonly linked across haplotype boundaries. Asterisks indicate maximally informative SNPs. (c) relative frequency of the different haplotypes in trios and their sum. (d) Degree of Linkage disequilibrium between the blocks estimated as d-prime. To conserve space, many statistically redundant SNPs were removed from the figure. For more details see FIG. 2 of Daly, Rioux et al. 2001.

FIG. 2. is a schematic describing the search for genes affecting human longevity. Before any genotyping began, it was important to demonstrate evidence that longevity runs in families (70) and, consequently, the prior probability of finding longevity-modulating genes was high. The subsequent linkage genome-wide scan focused attention on an extended region of chromosome IV (72). To identify the specific alleles involved, a haplotype map of the region was created using familial trios (74) and this map was used to identify a specific risk haplotype, as described herein. The study included a haplotype association study of long lived individuals compared to controls (76) and testing of associations with independent samples (78). Several rounds of SNP discovery (80), haplotype reconstruction, and mapping (82) were required to exhaust the search for potentially causative variants (84). Because MTP can only explain a small fraction of the total genetic variance in human longevity and there may be dozens of genes with a similar association, in the near future additional studies will likely yield an insight into the genetic basis of longevity, aging, and disease resistance.

Methods

Sample ascertainment and phenotyping. The study sample consists of individuals 98 years and older. Individuals were identified and recruited by a variety of methods including institutional websites, direct mailings and advertisement in newspapers geared towards potential participants or organizations involved with the aging community. Physical and cognitive health was not used as participation criteria. All participants and/or their legally authorized representatives took part in a written informed consent process. Additional collected data included health and socio-demographic histories, proof of age, usually in the form of a birth certificate, a three-generation pedigree and measures to assess functional independence and cognitive status.

Potential biases in the study may include subtle sample bias towards healthier study participants as a result of recruitment methods. For example, contact may result in part from the families of potential study participants with higher physical and cognitive status than the average nonagenarian/centenarian. This may explain the lower incidence of age associated diseases (i.e. cardiovascular disease, stroke) in the study group than expected. Controls (self-identified as “Caucasian” and less than 50 years of age) were obtained from several anonymous sources in the U.S. and Europe.

SNP validation. To screen this initial set of SNPs, 19 familial trios (mother, father, and offspring) acquired from the Centre d'Etude du Polymorphisme Humain (CEPH) Repository were genotyped at all selected markers. Of these 2000 markers, 1494 had high confidence calls on the MassArray™ platform. Of these markers, 990 had a minor allele frequency of at least 5%. SNPs of lower heterozygosity were excluded because of the reduced power of such markers with respect to mapping complex traits in association studies with limited sample size. Of the remaining SNPs, 113 were eliminated because the frequency distribution of the two types of homozygotes and heterozygotes as not statistically compatible with Hardy-Weinberg equilibrium. These failures were attributed to systematic artifacts introduced by the genotyping platform. The use of familial trios allowed a Mendelian check on the validity of each SNP assay. If more than one Mendelian inheritance error per assay was detected within the 19 trios, the assay was judged unreliable. Finally, of the 875 remaining SNPs, approximately 700 “maximally informative SNPs” were required to reconstruct all the identified haplotypes.

Genotyping. Potential SNPs were retrieved from the Human genome draft database. Assays were designed using spectroDESIGNER software (Sequenom, Inc.) to be multiplexed up to five times.

SNP genotyping was performed by Sequenom's chip-based matrix-assisted laser desorption/ionization time-of-flight (MALDI-TOF) mass spectrometry (DNA MassARRAY™) on PCR-based extension products from individual DNA samples. Cases and controls were always run on the same chip to avoid potential artifacts due to chip-specific miss-calls.

Sequencing. Samples homozygous with respect to the risk block were identified. Two homozygotes for each of the five haplotypes were selected for the sequencing of 84 kb spanning rs1553432 and the risk block. Sequencing was performed on the AB 3100 using a BigDye™ termination (version 3) chemistry on RapXtract (from Prolinks Inc.) purified PCR products. Phred program (by Codoncode) was used for quality scores and Sequencer (by Genecodes) for sequence comparisons and SNP detection.

Haplotype reconstruction. 19 familial trios (mother, father, offspring) were genotyped with densely spaced SNP markers in order to create a haplotype map of the 12 cM region. F or each trio, the parental origin of offspring alleles was determined for all cases where phase could be resolved unambiguously. In cases where phase was ambiguous (i.e. triple heterozygotes), the data were treated as missing. By applying this method, four parental chromosomes were reconstructed, with intermittent missing allele data. For this example, haplotypes were used that correspond to a region of DNA with little evidence (<2.5%) for meiotic recombination within the common genetic history of the individuals genotyped.

In situations where the boundaries were ambiguous, a second heuristic was applied that assigned boundaries in such as way to minimize the size (i.e. base pairs) within each block. With haplotype boundaries assigned, haplotype frequencies were estimated for each haplotype allele using an Expectation Maximization (EM) algorithm (Excoffier and Slatkin 1995). Any haplotype that had a frequency of less than 2.5% was excluded from further analysis to avoid possible errors in either the genotyping or the estimation process. Within each haplotype block, between 2 and 6 common SNP-based haplotypes were observed, and each of these haplotypes could be used as genetic markers.

In order to reconstruct haplotypes for the case/control association studies, the haplotype boundaries and allele frequency estimates established in the trios are used as initial parameters to seed the haplotype allele frequency estimations from genotyping the cases and controls. This seeding is important because of the significant amount of ambiguous phase information present in pairs of unrelated chromosomes. In cases where haplotype data could not be estimated with >95% confidence, the haplotype allele was treated as missing.

Tests of association. The G-Test with Williams correction (a statistic following a chi-square distribution) was used to test inferences about associating genetic markers (haplotype or SNP) with the longevity phenotype (Sokal and Rohlf 2000). For each allele, 2×2 contingency tables were constructed as +/−allele vs. +/−longevity. For tests where only one direction of allele frequency difference was tested, p values were divided by two. The Hotelling T test is the multivariate extension of the Student's T test and has recently been applied to genetic data (Xiong, Zhao et al. 2002).

Testing for stratification. 60 random SNP markers were genotyped in all cases and controls and chi-square values were calculated from the allele counts. Because these SNPs were selected at random, any differences in allele frequencies were inferred as representative of the differences in genetic backgrounds between cases and controls. If the genetic backgrounds of the two armed study were perfectly matched, the mean chi-square of the G-Test statistics for these markers have an expected value of 1.0.

Proactive sample matching. 60 random SNP markers (non-overlapping with the stratification panel described above) were genotyped in 250 cases and 463 controls. Homozygotes for the minor allele were assigned the value −1, heterozygotes 0, and major allele homozygotes 1. Based on the multivariate means calculated from this coded data, a subgroup of the 250 controls was selected that minimized the Mahalanobis distance with respect to the case samples. The Mahalanobis distance is a measure of distance between two multivariate means that normalizes each dimension based on the covariance matrix:

D=(\overline{V}₁−\overline{V}₂)S⁻¹(\overline{V}₁−\overline{V}₂)^{T},

where \overline{V}₁ is a vector representing the mean genotyping values of the cases, \overline{V}₂ is the mean vector for the controls, and S⁻¹ is the inverse of the covariance matrix.

### REFERENCES

1. Gudmundsson, H., Gudbjartsson, D. F., Frigge, M., Gulcher, J. R. & Stefansson, K. Inheritance of human longevity in Iceland. Eur J Hum Genet 8, 743-9 (2000).

2. Perls, T. et al. Exceptional Familial Clustering for Extreme Longevity in Human. J Am Geriatr Soc 48, 1483-1485 (2000).

3. Herskind, A. M. et al. The heritability of human longevity: a population-based study of 2872 Danish twin pairs born 1870-1900. Hum Genet 97, 319-23 (1996).

4. McGue, M., Vaupel, J. W., Holm, N. & Harvald, B. Longevity is moderately heritable in a sample of Danish twins born 1870-1880. J Gerontol 48, B237-44 (1993).

5. Perls, T. et al. Life-long sustained mortality advantage of siblings of centenarians. Proc Natl Acad Sci USA 99, 8442-8447 (2002).

6. van Bockxmeer, F. M. ApoE and ACE genes: impact on human longevity. Nat Genet 6, 4-5 (1994).

7. Schachter, F. et al. Genetic associations with human longevity at the APOE and ACE loci. Nat Genet 6, 29-32 (1994).

8. Arking, D. E. et al. Association of human aging with a functional variant of klotho. Proc Natl Acad Sci USA 99, 856-861 (2002).

9. Kervinen, K. et al. Apolipoprotein E and B polymorphisms—longevity factors assessed in nonagenarians. Atherosclerosis 105, 89-95 (1994).

10. Schachter, F. Causes, effects, and constraints in the genetics of human longevity. Am J Hum Genet 62, 1008-14 (1998).

11. Wachter, K. W. In Between Zeus and the Salmon. The Biodemography of Longevity (National Academy Press, Washington, D.C., 1997).

12. Puca, A. A. et al. A genome-wide scan for linkage to human exceptional longevity identifies a locus on chromosome 4. Proc Natl Acad Sci USA 98, 10505-8 (2001).

13. Patil, N. et al. Blocks of limited haplotype diversity revealed by high-resolution scanning of human chromosome 21. Science 294, 1719-23 (2001).

14. Stephens, J. C. et al. Haplotype variation and linkage disequilibrium in 313 human genes. Science 293, 489-93 (2001).

15. Johnson, G. C. et al. Haplotype tagging for the identification of common disease genes. Nat Genet 29, 233-7 (2001).

16. Daly, M. J., Rioux, J. D., Schaffner, S. F., Hudson, T. J. & Lander, E. S. High-resolution haplotype structure in the human genome. Nat Genet 29, 229-32 (2001).

17. Rioux, J. D. et al. Genetic variation in the 5q31 cytokine gene cluster confers susceptibility to Crohn disease. Nat Genet 29, 223-8 (2001).

18. De Benedictis, G. et al. DNA multiallelic systems reveal gene/longevity associations not detected by diallelic systems. The APOB locus. Hum Genet 99, 312-8 (1997).

19. Stephens, J. C. Single-nucleotide polymorphisms, haplotypes, and their relevance to pharmacogenetics. Mol Diagn 4, 309-17 (1999).

20. Davidson, S. Research suggests importance of haplotypes over SNPs. Nat Biotechnol 18, 1134-5 (2000).

21. Hirschhorn, J. N., Lohmueller, K., Byrne, E. & Hirschhorn, K. A comprehensive review of genetic association studies. Genet Med 4, 45-61 (2002).

22. Wetterau, J. R., Lin, M. C. & Jamil, H. Microsomal triglyceride transfer protein. Biochim Biophys Acta 1345, 136-50 (1997).

23. Shelness, G. S. & Sellers, J. A. Very-low-density lipoprotein assembly and secretion. Curr Opin Lipidol 12, 151-7 (2001).

24. Jamil, H. et al. Evidence that microsomal triglyceride transfer protein is limiting in the production of apolipoprotein B-containing lipoproteins in hepatic cells. J Lipid Res 39, 1448-54 (1998).

25. Wu, X., Zhou, M., Huang, L. S., Wetterau, J. & Ginsberg, H. N. Demonstration of a physical interaction between microsomal triglyceride transfer protein and apolipoprotein B during the assembly of ApoB-containing lipoproteins. J Biol Chem 271, 10277-81 (1996).

26. Berriot-Varoqueaux, N., Aggerbeck, L. P., Samson-Bouma, M. & Wetterau, J. R. The role of the microsomal triglygeride transfer protein in abetalipoproteinemia. Annu Rev Nutr 20, 663-97 (2000).

27. Raabe, M. et al. Knockout of the abetalipoproteinemia gene in mice: reduced lipoprotein secretion in heterozygotes and embryonic lethality in homozygotes. Proc Natl Acad Sci USA 95, 8686-91 (1998).

28. Tietge, U. J. et al. Hepatic overexpression of microsomal triglyceride transfer protein (MTP) results in increased in vivo secretion of VLDL triglycerides and apolipoprotein B. J Lipid Res 40, 2134-9 (1999).

29. Raabe, M. et al. Analysis of the role of microsomal triglyceride transfer protein in the liver of tissue-specific knockout mice. J Clin Invest 103, 1287-98 (1999).

30. Bjorkegren, J., Beigneux, A., Bergo, M. O., Maher, J. J. & Young, S. G. Blocking the secretion of hepatic very low density lipoproteins renders the liver more susceptible to toxin-induced injury. J. Biol Chem 277, 5476-83 (2002).

31. Wetterau, J. R. et al. An MTP inhibitor that normalizes atherogenic lipoprotein levels in WHHL rabbits. Science 282, 751-4 (1998).

32. Lin, M. C. et al. Garlic inhibits microsomal triglyceride transfer protein gene expression in human liver and intestinal cell lines and in rat intestine. J Nutr 132, 1165-8 (2002).

33. Lin, M. C. et al. Ethanol down-regulates the transcription of microsomal triglyceride transfer protein gene. Faseb J 11, 1145-52 (1997).

34. Wilcox, L. J., Borradaile, N. M., de Dreu, L. E. & Huff, M. W. Secretion of hepatocyte apoB is inhibited by the flavonoids, naringenin and hesperetin, via reduced activity and expression of ACAT2 and MTP. J Lipid Res 42, 725-34 (2001).

35. Karpe, F., Lundahl, B., Ehrenborg, E., Eriksson, P. & Hamsten, A. A common functional polymorphism in the promoter region of the microsomal triglyceride transfer protein gene influences plasma LDL levels. Arterioscler Thromb Vasc Biol 18, 756-61 (1998).

36. Couture, P. et al. Absence of association between genetic variation in the promoter of the microsomal triglyceride transfer protein gene and plasma lipoproteins in the Framingham Offspring Study. Atherosclerosis 148, 337-43 (2000).

37. Juo, S. H., Han, Z., Smith, J. D., Colangelo, L. & Liu, K. Common polymorphism in promoter of microsomal triglyceride transfer protein gene influences cholesterol, ApoB, and triglyceride levels in young african american men: results from the coronary artery risk development in young adults (CARDIA) study. Arterioscler Thromb Vasc Biol 20, 1316-22 (2000).

38. Ledmyr, H. et al. Variants of the microsomal triglyceride transfer protein gene are associated with plasma cholesterol levels and body mass index. J Lipid Res 43, 51-8 (2002).

39. St-Pierre, J. et al. Visceral obesity and hyperinsulinemia modulate the impact of the microsomal triglyceride transfer protein −493G/T polymorphism on plasma lipoprotein levels in men. Atherosclerosis 160, 317-24 (2002).

40. Talmud, P. J., Palmen, J., Miller, G. & Humphries, S. E. Effect of microsomal triglyceride transfer protein gene variants (−493G>T, Q95H and H297Q) on plasma lipid levels in healthy middle-aged UK men. Ann Hum Genet 64, 269-76 (2000).

41. Herrmann, S. M. et al. Identification of two polymorphisms in the promoter of the microsomal triglyceride transfer protein (MTP) gene: lack of association with lipoprotein profiles. J Lipid Res 39, 2432-5 (1998).

42. Rainwater, D. L. et al. A genome search identifies major quantitative trait loci on human chromosomes 3 and 4 that influence cholesterol concentrations in small LDL particles. Arterioscler Thromb Vasc Biol 19, 777-83 (1999).

43. Austin, M. A. et al. Candidate-gene studies of the atherogenic lipoprotein phenotype: a sib-pair linkage analysis of DZ women twins. Am J Hum Genet 62, 406-19 (1998).

44. Barzilai, N., Gabriely, I., Gabriely, M., Iankowitz, N. & Sorkin, J. D. Offspring of centenarians have a favorable lipid profile. J Am Geriatr Soc 49, 76-9 (2001).

45. Terry, D., Wilcox, M., McCormick, M., Lawler, E. & Perls, T. Cardiovascular Advantages Among the Offspring of Centenarians. Journal Gerontological Medical Science In Press (2003).

46. Glueck, C. J., Gartside, P. S., Mellies, M. J. & Steiner, P. M. Familial hypobeta-lipoproteinemia: studies in 13 kindreds. Trans Assoc Am Physicians 90, 184-203 (1977).

47. Silverman, J. M. et al. Identifying families with likely genetic protective factors against Alzheimer disease. Am J Hum Genet 64, 832-8 (1999).

48. Excoffier, L. & Slatkin, M. Maximum-likelihood estimation of molecular haplotype frequencies in a diploid population. Mol Biol Evol 12, 921-7 (1995).

49. Sokal, R. R. & Rohlf, F. J. Biometry (W. H. Freeman and Company, New York, 2000).

50. Xiong, M., Zhao, J. & Boerwinkle, E. Generalized T2 test for genome association studies. Am J Hum Genet 70, 1257-68 (2002).

Other embodiments are within the following claims.

I claim:

1. A method comprising:
receiving information for each individual of a first group of individuals and each individual of a second group of individuals, wherein the information for each individual comprises indications about a plurality of different biological features;
selecting a subset of individuals from the second group using a comparison between information for members of the first group and information for members of the subset; and
evaluating the relationship of at least one factor to members of the first group relative to members of the selected subset.

2. The method of claim 1, wherein the different biological features comprises a property of a biomolecule.

3. The method of claim 2 wherein the biomolecule is a protein, nucleic acid, lipid, or carbohydrate.

4. The method of claim 3 wherein the different biological features comprise polymorphisms of genomic DNA.

5. The method of claim 1 wherein the different biological features comprises a property of a cell.

6. The method of claim 1 wherein the plurality of different biological features comprises at least ten features.

7. The method of claim 1 wherein the comparison comprises representing the information for each member as a multi-dimensional vector or matrix.

8. The method of claim 1 wherein the comparison is weighted by covariance of at least two different features.

9. The method of claim 7 wherein the comparison is weighted by a covariance matrix for the plurality of different features.

10. The method of claim 4, wherein the individuals are humans, the first group of individuals is associated with a particular phenotypic trait, and the evaluating comprises evaluating association of a genetic marker with individuals of the first group relative to individuals of the select subset.

11. The method of claim 10 wherein the plurality of different biological features comprises genetic polymorphisms located on at least four different chromosomes.

12. The method of claim 10 wherein the comparison comprises assessing a multivariate distance.

13. The method of claim 11 wherein the evaluating association of the genetic marker comprises evaluating a LOD score.

14. A method of evaluating the relationship between a genetic polymorphism and a trait, the method comprising:
obtaining nucleic acid from each individual of a plurality of individuals, wherein a first group of the individuals are associated with a trait and a second group of individuals are not associated with the trait;
analyzing the nucleic acid to determine genetic information about a plurality of genetic loci for each individual of the plurality;
selecting a subset of individuals from the second group based on a comparison between the genetic information for members of the first group and the genetic information for members of the subset; and
evaluating association of a genetic locus of interest and individuals of the first group relative to association of the genetic locus of interest and individuals of the selected subset.

15. The method of claim 14 wherein the genetic information comprises indications of presence or absence of single nucleotide polymorphisms at least some genetic loci.

16. The method of claim 14 wherein the selecting comprises selecting a subset that compares to the first group more favorably than at least another subset.

17. The method of claim 14 wherein the selecting comprises incrementally adding members of the second group to the subset.

18. The method of claim 17 wherein the incremental adding comprises selecting one or more members of the second group based on how a group that includes the one or more members compares to the first group.

19. The method of claim 18 wherein the incremental adding comprises selecting a single member of the second group that minimizes a comparative function for a comparison between a group that includes the single member and the first group.

20. The method of claim 14 wherein the comparison comprises a comparative function that returns a scalar value.

21. The method of claim 20 wherein the selecting comprises minimizing the comparative function.

22. The method of claim 20 wherein the comparative function is a function of distance.

23. The method of claim 22 wherein the distance is weighted for allele variability.

24. The method of claim 22 wherein the distance is weighted for allele co-variance.

25. The method of claim 22 wherein the distance is a Mahalanobis distance.

26. The method of claim 14 wherein the selecting comprises pairing each member of the first group to a unique member of the second group.

27. The method of claim 14 wherein the evaluating of the association comprises evaluating a LOD score for the marker of interest.

28. The method of claim 14 wherein the plurality of genetic markers excludes the marker of interest.

29. The method of claim 14 wherein the plurality of genetic markers contains between 10 and 100 markers.

30. The method of claim 25 wherein the selecting comprises a filter that requires that the mean chi-square of the G-test is less than 1.5.

31. A system comprising:
a memory that stores information for each individual of a first group of individuals and each individual of a second group of individuals, wherein the information for each individual comprises indications about a plurality of different biological features;
a communications interface;
and a processor configured to
select a subset of individuals from the second group using a comparison between information for members of the first group and information for members of the subset;
evaluate the relationship of at least one factor to members of the first group relative to members of the selected subset; and
communicate results of the evaluation using the interface.

32. A method comprising:
obtaining nucleic acid samples from each individual of a first group of individuals and each individual of a second group of individuals;
analyzing the nucleic acid samples to determine information about a plurality of genetic markers for each individual of the first and second groups;
selecting a subset of individuals from the second group using a comparison between the information for members of the first group and the information for members of the subset; and
comparing members of the first group to members of the selected subset with respect to at least one factor.

33. The method of claim 32 wherein the comparing comprise subjecting members of the first group, but not the second group to a condition and evaluating members of the first group and members the second group.

34. The method of claim 33 wherein the condition is a medical procedure.

35. The method of claim 32 wherein the comparison comprises a distance function that returns a scalar value.

36. The method of claim 35 wherein the distance function is weighted for marker co-variance.

37. A method comprising:
obtaining DNA samples from and information about each individual of a first group of the individuals are associated with a trait;
analyzing the DNA samples to determine genetic information about a plurality of genetic loci for each individual of the plurality;
sending the allelic information to a server that stores genetic information for each individual of a second group of individuals; and
receiving information about a subset of individuals selected from the second group of individuals, wherein the subset of individuals is selected using a comparison between the genetic information for members of the first group and genetic information for members of the selected subset.

38. A server comprising
a memory that stores allelic information for a plurality of genetic markers for each individual of a first group of individuals; and
software configured to:
receive genetic information about a plurality of genetic loci for each individual of a plurality of individuals;
select a subset of individuals from the second group using a comparison between genetic information for members of the plurality of individuals and genetic information for members of the selected subset; and
communicate information about individuals of the subset to a user.

39. A method of comparing a first and second population of individuals, the method comprising:
receiving genetic information for the first and second populations of individuals, the genetic information including information about a plurality of genetic markers for each of the individuals, the plurality including markers located on at least four different chromosomes and at least twenty different markers; and
returning a scalar value that is a function of the marker distribution for the first and second population and the degree of covariance among the genetic markers.

40. The method of claim 39 wherein the function further weights each marker by the degree of variability of the respective marker.

41. The method of claim 40 wherein the function is a function of the Mahalanobis distance between the genetic information for the first and second populations.

42. The method of claim 39 wherein each allele is weighted by its allele frequency in a third population.

43. A method of performing a controlled study, the method comprising:
identifying a first and second subset of individuals from the plurality of individuals by comparing occurrences of a plurality of genetic markers among individuals of the first and second subsets; and
subjecting the first subset of individuals to a first condition and the second subset of individuals to a second condition.

44. The method of claim 43 wherein the plurality of genetic markers includes markers located on at least four different chromosomes and at least twenty different markers.

45. The method of claim 44 wherein the first conditions comprises administering a test treatment, and the second condition comprises administering a control/placebo treatment.

46. The method of claim 43 wherein the comparing comprises evaluating a function that returns a scalar value and depends on of the marker distribution for the first and second subset and the degree of covariance among the genetic markers in the respective subsets.

47. The method of claim 44 where the subsets are complementary.

48. A machine readable medium having encoded thereon information comprising:
a first list of records;
a second list of records, wherein each record of the first and second list corresponds to a genome and comprises genetic information about each of a plurality of genetic markers in the genome; and
information describing a relationship between records of the first list and records of the second list, wherein the relationship is a function of the genetic information for at least a subset of the genetic markers, the markers of the subset including markers on at least two different chromosomes, and covariance of genetic markers of the subset between records of each list.

49. The medium of claim 47 wherein the relationship is a function of distance.

50. The medium of claim 48 wherein the distance is a Mahalanobis distance.
